(19) United States
(12) Patent Application Publication
(10) Pub. No.: US 2021/0228107 A1
(43) Pub. Date: Jul. 29, 2021

(54) SYSTEMS AND METHODS FOR CHARACTERIZATION OF JOINTS

(71) Applicant: THE BIONICS INSTITUTE OF AUSTRALIA, East Melbourne (AU)

(72) Inventors: Thushara Perera, East Melbourne (AU); Nichlas Sinclair, East Melbourne (AU); David Angus Begg, East Melbourne (AU)

(21) Appl. No.: 17/052,759

(22) PCT Filed: May 3, 2019
(86) PCT No.: PCT/AU2019/050408

§ 371 Date: Nov. 3, 2020

(30) Foreign Application Priority Data
May 4, 2018 (AU) 2018901532

(51) Int. Cl. A61B 5/11; A61B 5/00

(52) U.S. Cl. CPC A61B 5/1101; A61B 5/4082; A61B 2562/0219; A61B 5/4842; A61B 5/6826; A61B 5/4528

(57) ABSTRACT

A system, method and device for characterising a joint between a first body region and a second body region is disclosed. The system may comprise a reference portion configured to be coupled to the first body region, a movable portion configured to be coupled to the second body region and a movement source configured to move the movable portion relative to the reference portion, such as in a direction of the joint. A set of one or more sensors may collect a set of one or more measurements related to the movement between the reference portion and the movable portion, for example to determine a force between the movable portion and the body region coupled thereto, and/or a power consumed by the movement source. The measurements may be used to characterise the joint, such as by determining a rigidity of the joint.

### CROSS-REFERENCE TO RELATED APPLICATION

The present application claims priority to Australian provisional patent application no. 2018901532, filed on 4 May 2018, the entire content of which is herein incorporated by reference

### TECHNICAL FIELD

The present disclosure relates to systems, devices and methods for characterisation of joints. For example, the present disclosure may be relevant to measurement and assessment of joint rigidity, such as for diagnosis and/or titration for Parkinson's Disease or arthritis.

### BACKGROUND

A number of conditions and/or diseases may affect one or more characteristics of its patient(s). Conversely, severity of a patient's symptoms may be assessed to characterise a state of a disease and/or condition.

One such characteristic which has been measured is a rigidity of a joint. A clinician may be interested in measuring a rigidity, which may be defined by some clinicians as a resistance to passive movement. For example, a clinician may diagnose a disease and/or titrate therapy based at least partially on such a measurement.

Some of those with Parkinson's Disease (PD) may typically experience symptoms of tremor, rigidity, postural instability and/or bradykinesia (slowness). Such patients may experience an increase in rigidity according to a corresponding increase in muscle tone (co-contraction of extensor and flexor muscles), and measurements of such a change in rigidity may be useful indicators for the clinician.

In the example of PD, rigidity is common to its patients, and it is often one of the first symptoms to be affected by changes in therapy or progression of disease. As such, accurate assessment of rigidity may be useful for monitoring disease progression or assessing efficacy of therapy. Quantification of rigidity may be advantageous, for example: during clinical consultations for diagnosis and management of PD; in clinical trials where new therapeutic interventions are investigated; and/or in research to determine mechanistic insights about pathophysiology.

The severity of PD symptoms have been assessed using a clinical rating scale, such as the Movement Disorder Society's Unified Parkinson's Disease Rating Scale (UPDRS, or MDS-UPDRS), which requires a movement disorder specialist grading or scoring symptoms on a 0-4 ordinal scale based on observation. In this method, clinicians manipulate the patient's limbs and subjectively gauges the force required to complete movements along the full range of joints using the instructions given in the UPDRS, wherein the resulting assessments are used to diagnose PD as well as to titrate therapy.

However, such methods may suffer from one or more inherent problems. They include (in no particular order): assessment variability due to subjective clinical assessment and/or variability due to sampling, lack of sensitivity, and requirement for a movement disorder specialist to be present.

Subjective nature of the above clinical assessment may also cause secondary difficulties. Namely, recruitment of one or more independent movement disorder specialists may be expensive, time consuming and/or tiring for the patient, particularly if repetitions are required for multiple specialists, and scheduling challenges may arise in potentially recruiting multiple specialists if required to do so for a clinical trial. Another potential risk is that there may not be sufficient resolution in the five-point 0-4 UPDRS scale, preventing for instance detection of small changes in severity of the symptom. This may not only increase difficulties and frustrations for the patient, but also may be concerning for researchers trialing new interventions.

Also, it is possible for symptoms of PD to fluctuate in severity due to medication pharmacokinetics, psychological factors (e.g. stress, attention, fatigue) and other factors such as the circadian rhythm. Therefore, a limited sampling of data, such as a measurement during a single visit to the clinician, may not provide sufficient information to provide therapeutic recourse.

A number of efforts have been made in order to better determine rigidity in joints (e.g. in human joints), such as a pendulum-based system to elicit repeatable forced-flexion tasks in participants while measuring forces on the limb with specialised transducers. Such efforts could be potentially categorised broadly as either automated or semi-manual, wherein automated approaches use an electric motor to drive flexion of the limb (e.g. a wrist) in a repeatable manner with displacement and force/torque being output measures. Semi-automatic approaches may include an investigator manipulating the participant's limb via a force transducer in time to a visual cue or audible metronome.

Such approaches have not been without problems. For one, relatively large motors may be required to provide enough torque at sufficient speeds to flex the joint, which may not only cause the patient apprehension but also limit its utility in hospital environments due to the noise produced. Also, patients may try to actively assist the device, thereby confounding the results found. Semi-manual methods are prone to increased variability while they may not solve the problem of bias.

Other approaches, such as electromyography (EMG), have also been investigated but have not shown much promise due to confounding elements such as motion artefact and variability across subjects.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### SUMMARY

Certain aspects of the present disclosure relate to systems, devices and methods for characterising a joint, such as a wrist, elbow, ankle, knee or a finger joint. In some examples, a system may comprise a reference portion and a movable portion, connected to and configured to be moved relative to each other by a movement source. The reference portion may be coupled to a first region of the body such as a palm or a forearm, and the movable portion may be coupled to a second region of the body, such as a finger or palm respectively, e.g., to characterise a metacarpophalangeal joint or wrist respectively.

Characterisation of a joint, as referred to in the present disclosure, may relate to determining of a rigidity, such as a rigidity of a wrist, and/or to determining other joint parameters or behaviours such as tremor, bradykinesia or instability. It should be understood that where a ‘joint rigidity’ is referred to, it may not necessarily be limited to a rigidity of the ‘joint’ in isolation, but may refer to a resistance to movement (e.g. in rotation) of one body region with respect to another (e.g. a first body region with respect to a second body region). In other words, a ‘wrist rigidity’ may simply refer to a resistance to passive movement of a hand with respect to the adjacent forearm. As an example, an ‘elbow rigidity’ may be a function of a condition of one or muscle groups of the forearm, as well as a function of a condition of the elbow region itself.

The system may comprise a set of one or more sensors configured to collect a set of one or more measurements related to the movement between the reference portion and the movable portion, while the system is placed in situ.

Following from this, in one aspect of the present disclosure there is provided a system for characterising a joint between a first body region and a second body region, the system comprising a reference portion configured to be coupled to the first body region; a movable portion configured to be coupled to the second body region and to allow the second body region to move relative to the first body region in a first direction when coupled thereto; a movement source configured to move the movable portion relative to the reference portion in the first direction; a set of one or more sensors configured to collect a set of one or more measurements related to the movement between the reference portion and the movable portion, and a processor, for characterising the joint based on the one or more measurements.

Further, one aspect of the present disclosure relates to a method for characterisation of a joint between a first body region and a second body region, the method comprising: providing a reference portion coupled to the first body region; providing a movable portion coupled to the second body region; providing a movement source configured to move the movable portion relative to the reference portion in a movement direction of the joint; performing a movement of the first body region relative to the second region in the movement direction, collecting, using a set of one or more sensors, measurements related to the movement between the reference portion and the movable portion and characterising the joint based on the one or more measurements.

Further, one aspect of the present disclosure relates to a device for characterising a joint between a first body region and a second body region, the device comprising: a reference portion configured to be coupled to the first body region; a movable portion configured to be coupled to the second body region and to allow the second body region to move relative to the first body region in a first direction when coupled thereto; a movement source configured to move the movable portion relative to the reference portion in the first direction; and a set of one or more sensors configured to collect a set of one or more measurements related to the movement between the reference portion and the movable portion.

The set of measurements may be used to determine a torque required to move the movable portion and/or a force applied by the finger during said movement. The torque and/or the force determined may be then used to characterise the joint, e.g., by producing a clinical rating. Using this system, internally-induced movement or effort (e.g. patient-initiated effort) may be accounted for, which otherwise may reduce the accuracy of the characterisation.

In some embodiments, the torque may be determined from power consumed by the movement source. In some embodiments, the power consumed may be determined based on a measurement of a current draw of the movement source.

The set of sensors may include one or more of a force transducer, a current transducer, a displacement transducer and an inertial motion unit. The set of sensors may produce a set of measurements for a processor to produce a clinical rating therefrom. The processor may produce the clinical rating using one or more of lookup tables and/or transfer functions, wherein the lookup tables and/or transfer functions may be derived using a regression model. The lookup tables and/or transfer functions may be predetermined, such as by being retrievably saved in memory to be accessed by the processor.

One aspect of the present disclosure relates to a system for characterising a rigidity of a joint between a first body region and a second body region, the system comprising a reference portion configured to be coupled to the first body region; a movable portion configured to be coupled to the second body region and to allow the second body region to move relative to the first body region in a first direction when coupled thereto; a movement source configured to move the movable portion relative to the reference portion in the first direction; a set of sensors comprising: a first sensor configured to determine a force between the movable portion and the second body region; a second sensor configured to determine a power consumed by the movement source; and a processor, wherein the processor is configured receive a set of signals from the set of sensors indicative of determinations from its sensors and to deliver an output signal indicative of the rigidity of the joint based at least on the set of signals.

The joint may be a metacarpophalangeal joint. The set of sensors may further comprise a third sensor configured to determine a position of the movable portion relative to the reference portion. The set of sensors may further comprise a fourth sensor configured to determine inertial motion of the system. The output signal may comprise a clinical rating. The processor may determine the clinical rating based on a lookup table. The output signal may comprise one or more of: a force rate, peak force, work, peak current and charge. The processor may be configured to discard a portion of the set of signals based on predetermined criteria. The predetermined criteria may include a threshold rate of change of output of the first sensor.

One aspect of the present disclosure relates to a method for determining a rigidity of a joint between a first body region and a second body region for characterisation of the joint, the method comprising: providing a reference portion coupled to the first body region; providing a movable portion coupled to the second body region; providing a movement source configured to move the movable portion relative to the reference portion in a movement direction of the joint; performing a movement of the first body region relative to the second region in the movement direction, wherein a set of sensors delivers a set of sensor outputs indicative of a force between the movable portion and the second region and a power consumed by the movement source; and determining, using a processor, a rigidity of the joint at least partly based on the sensor outputs.

The joint may be a finger joint. The rigidity of the joint may be determined using a lookup table. he movement may comprise a plurality of flexion cycles of the joint. The movement may consist of between 20 to 40 flexion cycles. The joint rigidity may be determined as a clinical rating. The set of sensor outputs may be further indicative of tremors occurring on the joint. The set of sensor outputs may be further indicative of a position of the movable portion. The set of sensor outputs may be based on measurements at 100 Hz. The movement may be at a substantially constant speed.

One aspect of the present disclosure relates to a device for characterising a rigidity of a joint between a first body region and a second body region, the device comprising: a reference portion configured to be coupled to the first body region; a movable portion configured to be coupled to the second body region and to allow the second body region to move relative to the first body region in a first direction when coupled thereto; a movement source configured to move the movable portion relative to the reference portion in the first direction; a first sensor configured to determine a force between the movable portion and the second body region; and a second sensor configured to determine a power consumed by the movement source.

In some forms of the system, method or device, according to aspects of the present disclosure, the first sensor is located on a surface of the movable portion and/or the movable portion is configured to be coupled to a finger and the reference portion is configured to be coupled to a palm.

In some forms of the system, method or device, according to aspects of the present disclosure, the reference portion may be configured to be coupled to the palm of a patient's hand, while the movable portion may be configured to be coupled to the patient's second finger. The reference portion may include a palm support coupled to the palm of the patient's hand and one or more finger support extensions that extend from the palm support. A gap may be present between the finger support extensions through which the patient's second finger and/or movable portion can be moved. One of the finger supports may support the first finger of the patient. One of the finger supports may support the third and fourth fingers of the patient. One or more securement devices may be provided to hold the fingers immobile relative to the reference portion. The one or more securement devices may comprise hook-and-loop fasteners, adhesive, tape, sleeves, straps, locking pins, ratchet and pawl mechanism and/or other suitable securement mechanisms.

Throughout this specification the words “comprise”, “include” and “have”, and variations such as “comprises”, “comprising”, “includes”, “including”, “has” and “having”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

Embodiments of the present disclosure will now be described, by way of example only, with reference to the accompanying drawings, in which:

FIG. 1 shows a perspective view of system according to an embodiment of the present disclosure;

FIG. 2 shows a graph of validation study results, plotting predicted data determined from outputs of a system according to an embodiment of the present disclosure, plotted against a validation data set of clinical ratings according to MDS-UPDRS, and an overlaid linear regression graph;

FIG. 3 shows a front-on view of a system according to an embodiment of the present disclosure in-situ, wherein a movable portion is coupled to the second finger of a user, and a reference portion is coupled to a palm of the user;

FIG. 4 shows a side-on view of the system shown in FIG. 3;

FIG. 5 shows an example block diagram outlining interconnectivities of a system according to an embodiment of the present disclosure;

FIG. 6 shows an example graph showing measured force data during operation of a system embodiment of the present disclosure;

FIG. 7 shows an example graph showing measured current data during operation of a system embodiment of the present disclosure;

FIG. 8 shows a perspective view of a system according to another embodiment of the present disclosure;

FIG. 9 shows a top view of the system of FIG. 8;

FIG. 10 shows a series of column graphs of Force Rate and Work Estimate used as stand-alone measures to indicate rigidity on and off deep brain stimulation therapy for Parkinson's Disease and control groups; and

FIGS. 11A to 11C show Force Rate, Work Estimate and a clinical rigidity rating, respectively, measured off deep brain stimulation (indicated by the grey shaded region) and on deep brain stimulation.

### DESCRIPTION OF EMBODIMENTS

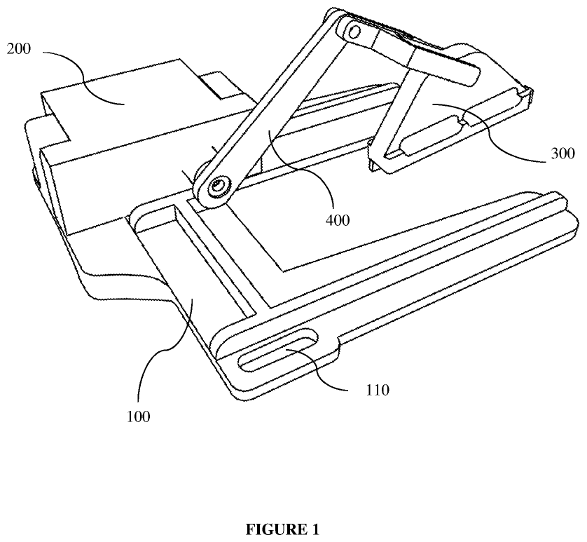

FIG. 1 shows a system according to an embodiment of the present disclosure, comprising a reference portion 100, such as a rigid polymer body, a movement source 200, such as a motor, a movable portion 300 and a connecting portion 400. FIG. 1 further shows the movable portion 300 and the connection portion 400 to be pivotably connected to each other. The reference portion 100 is configured to be coupled to a first body region, such as a palm of a hand, and the movable portion 300 is configured to be coupled to a second body region, such as a second finger of the same hand.

The system may be configured to allow a movement of the first body region in relation to the second body region in order to characterise a joint that comprises and/or is located between the first body region and the second body region. When the joint is a metacarpophalangeal joint of a finger, for example, the movement may include flexion and extension of the finger relative to the palm and may ultimately be used to determine the rigidity of the metacarpophalangeal joint. As another example, the joint may be a wrist, and the movement of flexion of the hand relative to the forearm may be used to characterise a rigidity of the wrist. In FIG. 1, the connecting portion 400 is rotationally coupled to the reference portion 100 and the movable portion 300, and the movement source 200 is rotationally coupled to the connecting portion 400, providing the ability to drive the flexion as required.

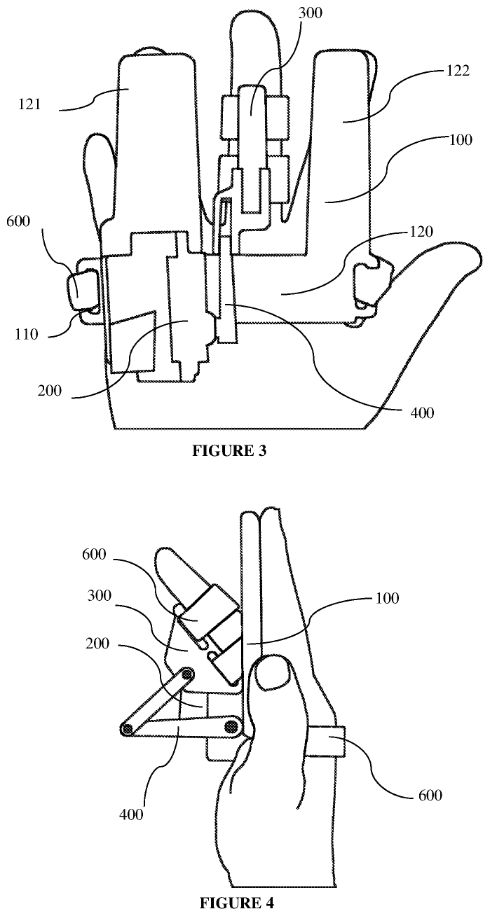

In the illustrated embodiment, the reference portion 100 is configured to be coupled to the palm of a patient's hand, while the movable portion 300 is configured to be coupled to the patient's second finger. As can be seen in FIGS. 3 and 4, the reference portion 100 lies substantially flat against the patient's palm. In this embodiment, the reference portion includes a palm support 120 coupled to the palm of the patient's hand and a pair of finger support extensions 121, 122 extending from the palm support 120. One of the extensions supports the first finger of the patient while the other extension supports the third and fourth fingers of the patient. A gap is present between the finger support extensions through which the patient's second finger and/or the movable portion 300 can be moved. The supported fingers are coupled to the extensions 121, 122 such that they are immobile relative to the reference portion 100. As shown in FIG. 3, the support 121 for the third and fourth fingers is approximately twice the width of the support 122 for the first (or index) finger. These finger support extensions may stabilise the reference portion and prevent rocking or wobbling of the reference portion when the movable portion 300 is moved by the movement source 200, thus increasing the accuracy of measurements obtained by the system.

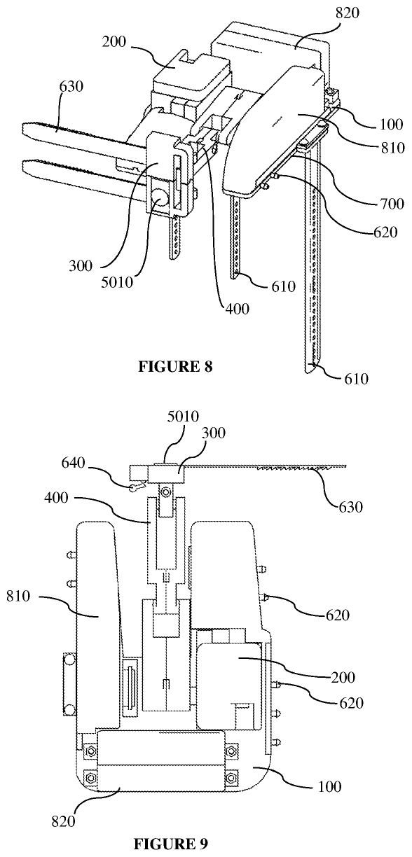

The reference portion 100 and the movable portion 300 may be coupled to the first and second body region respectively in any number of different ways. For instance, they may be coupled using one or more securement devices 600. In some embodiments, the securement devices may be hook-and-loop type fasteners. The hook-and-loop fasteners may tie the reference portion 100 to the first body region and the movable portion 300 to the second body region. Alternatively, or additionally, other means may be used, such as adhesive, tape (e.g. double-side tape) or sleeves. For example, a bio-compatible, soluble adhesive may be used for temporary bonding of the reference portion 100 to the first body region. An outer sleeve may be bonded to the movable portion 300 for insertion of the second body region thereto. In another example, as shown in FIGS. 8 and 9, the reference portion 100 may be coupled to the first body region by a securement device comprising adjustable flexible straps 610 with locking pins 620 for securing the straps. This configuration may: allow the system to fit a greater range of body region sizes; provide for quicker application and removal of the system; render the system more robust against wear and tear; and/or allow for easier disinfection or cleaning of the system after use.

In this embodiment, the movable portion 300 is secured to the second finger via a quick-release connector, including a linear ratchet-type adjustment mechanism 630 and a releasable pawl mechanism 640. The ratchet adjustment mechanism 630 allows the straps to be tightened to ensure secure fastening of the movable portion to the second body region and provides for easy and precise adjustment of clamping force. The quick-release pawl mechanism 640 allows the device to be removed from the second body region quickly and easily.

As shown in FIG. 8 the system may further comprise a comfort underlay 700 to provide cushioning between the reference portion 100 and the first body region. The underlay may be removable, for example, to be cleaned and re-used. In other embodiments, the underlay may be a single use disposable item. In other embodiments, the underlay 700 may be irremovably fixed to the reference portion 100. The underlay 700 may comprise, for example, a silicone gel, a foam layer or a layer of an alternative material suitable for providing a cushioning effect. The comfort underlay may increase the comfort of a patient in contact with the system by reducing pressure between the reference portion and the first body region. This may reduce patient fatigue and thus allow the system to be used for longer periods of time.

In the embodiment illustrated in FIGS. 8 and 9 the system further comprises a battery compartment 810 for housing an on-board battery power source and an electronics compartment 820 for housing on-board electronics. In this embodiment, the battery compartment 810 is positioned on or provides a portion of the reference portion 100 including the finger support extension 122 for the first (index) finger, while the electronics compartment 820 is positioned on or provides a portion of the reference portion 100 including the palm support portion 120.

The system may be operated wirelessly, resulting in improved workflow and freedom of movement for both the patient and the clinician.

The system may further comprise a set of one or more sensors, the set of sensors for example configured to produce a set of one or more signals indicative of measurements from each sensor.

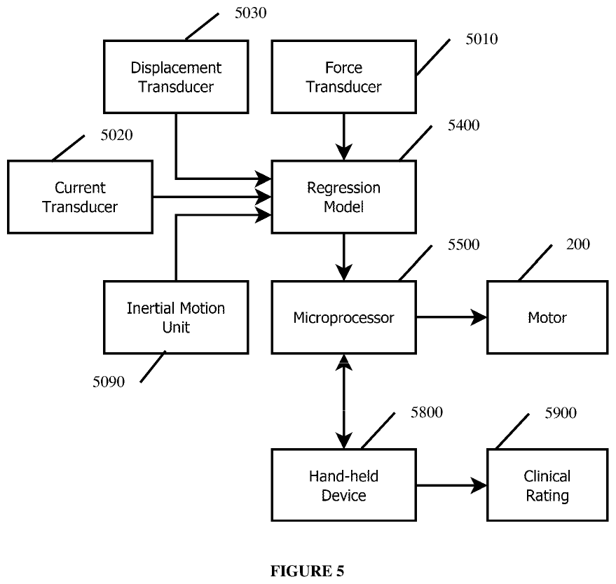

The system may comprise a force sensor configured to determine a force between the movable portion 300 and the second body region. The force sensor may be a force transducer 5010 as shown in FIG. 5, and may be a force sensing resistor, a strain sensor, or a pressure sensor. The force sensor may be configured to determine the force in a direction along the relative movement of the first body region and the second body region. In one example, the force sensor may be located on a surface of the movable portion 300, such that it would be configured to come into contact with the second finger.

The system may comprise a displacement sensor configured to determine a position of the movable portion 300, such as relative to the reference portion 100. The displacement sensor may be a displacement transducer 5030 as shown in FIG. 5, but could be a proximity sensor, optical sensor, rotary encoder or a Hall Effect sensor. The displacement sensor may be located at least partly on the movable portion 300, e.g. mounted on the movable portion 300 and the reference portion 100; however, any number of other arrangements may be suitable.

The system may also be configured to determine a power consumed by the movement source 200, such as by measuring a current draw of the motor. In one arrangement, the system may comprise a power sensor to determine the power consumed by the movement source 200. The power sensor may for example be a current transducer 5020 as shown in FIG. 5, and may be separate to the movement source 200, or may be an integral part thereof. For instance, where the movement source 200 is an electrical motor, the power sensor may monitor the current draw of the motor.

One or more of the set of sensors may be configured to deliver output data indicative of the properties being measured. For instance, the force transducer 5010 may deliver an output indicative of the force applied to the movable portion 300, the displacement transducer 5030 may deliver an output indicative of the displacement of the movable portion 300 and the current transducer 5020 may be configured to deliver an output indicative of the power consumed by the motor.

In some forms, one or more sensors may be substituted for by another input in order to approximate, or estimate the property in question. In one example, the displacement sensor may be substituted for by an output of the motor, wherein a position of the movable portion 300 may be inferred from one or more outputs of the motor.

Each of the set of sensors may be configured to record data at one common frequency, or at different frequencies. Each frequency may be above a threshold frequency, in order to ensure that sufficient data is captured throughout the flexion cycles. As an example, the threshold frequency may be set at 100 Hz and each sensor of the system may be configured to sample at 250 Hz. In some embodiments, a sampling frequency may be set based on a speed of system operation, such as the rate at which the joint is moved.

It is possible that, for at least some patients, a rigidity of a joint may vary according to a speed at which the joint is operating. The system may be configured to operate the movable portion 300 (and the corresponding body region) at a substantially constant speed to obtain consistent, controlled results. One suitable speed of operation of the system may be approximately 1 Hz, or one flexion cycle per second. In some forms, the suitable substantially constant speed may be defined as an absolute velocity (linear or angular) rather than as a frequency.

The reference portion 100 may be configured to retain the first body region in a relatively steady configuration throughout a flexion cycle. The reference portion 100 may comprise a relatively rigid material such as a plastic (e.g. polycarbonate) material. The reference portion 100 may comprise one or more stiffening ribs 101 as shown in FIG. 1, or any number of other strengthening features. The reference portion 100 need not be wholly rigid. In some forms, the reference portion 100 may comprise flexible or soft components, such as for improved usability, while providing a reference point for measurements and/or allowing component mounting thereto. Although element 100 provides a reference portion, the system may adopt or utilise one or more other elements as a reference portion. In some embodiments, the reference portion may comprise multiple components.

The reference portion 100 may comprise a set of attachment points, such as for anchoring relative to the first body region (e.g. a palm or a forearm) and/or the movement source 200, for example a motor housing or mount. For example, the reference portion 100 may comprise a set of slots 110 as shown in FIG. 1, each configured to receive a securement device 600 (for example, as shown in FIGS. 3-4). As described above, the securement device 600 may be a hook-and-loop fastener, however a number of other fastener types may be suitable, as may be other arrangements of securement or coupling.

The movement source 200 may be a motor, such as a servo motor typically used in robotics. The motor may be configured to provide a range of torques and speeds, suitable to drive the second body region (e.g. a finger, or hand), as well as preferably to allow precise positioning, while limiting noise output. As described further elsewhere in the present specification, the movement source 200 may be configured to drive the movable portion 300 at a substantially constant speed, in order to determine a joint rigidity on a consistent basis. In addition to servo motors, a number of other types of motors, or types of movement sources, may be suitable.

In some embodiments, such as in that shown in FIG. 1, the system is configured for characterisation of a finger joint. In such embodiments, relatively small and quiet motors may be suitable. Such embodiments may be particularly advantageous in environments such as hospitals, or for domestic use, where size and noise is a concern. Further, such embodiments may help to achieve wider adoption from patients by reducing user apprehension and disruption. Embodiments of the system may be used to collect data over an extended period of time due to improved usability and reduced obtrusiveness.

The system may further comprise a processor (e.g. microprocessor 5500 shown in FIG. 5) configured to receive, determine and/or deliver one or more outputs indicative of the joint rigidity. The one or more outputs may be based, at least in part, on measurements from the force transducer 5010, displacement transducer 5030 and/or current transducer 5020. The sensor outputs may indicate a level of resistance to movement of the joint. This resistance to movement may be measured in terms of one or more of force rate, peak force, work, peak current or charge.

In some embodiments, at least one output may be a clinical rating 5900 as shown in FIG. 5. The output may be communicated through a hand-held output device 5800 such as a smartphone or a tablet. The output may be communicated in one or more forms, including but not limited to: an audio signal, text message, e-mail, or electronic signal. In some embodiments, the output may be sent to an output device such as a monitor, a remotely located computer or a printer.

Determination of the output may utilise one or more additional inputs, such as from an inertial motion unit (IMU) 5090 as shown in FIG. 5. The IMU 5090 may determine one or more of a specific force and angular movement rate of the system, for example, to detect any tremors present in the patient's hand. Some PD patients may exhibit tremors, which may affect readings related to rigidity. One or more outputs from the IMU 5090 may be used as inputs by the processor to filter out and thereby account for tremors. This may allow for a more accurate determination of joint rigidity than otherwise possible. For example, the one or more outputs of the IMU 5090 may be subtracted from measurements from other sensors such as the force transducer 5010 or from the current transducer 5020.

An example method of use of an embodiment of a system according to the present disclosure is as follows. The system may be coupled to a finger joint to be characterised, for example by strapping the movable portion 300 to the finger of the user and strapping the reference portion 100 to the palm of the user, as shown in FIGS. 3-4. At this point, the patient may manipulate their hand to a starting position, such as in an open-palmed posture. Then, the clinician may initiate the experiment with a controller, whereby the processor may begin to operate the motor 200 to move the movable portion 300 through a range of motion of the joint. The joint may be moved at a substantially constant rate, for example, 1 flexion cycle per second (1 Hz).

As the movable portion 300 is moved along the range of motion of the joint, the set of one or more sensors may record and/or output the set of measurements The measurements may be indicative of force between the finger and the movable portion 300, torque applied by the motor 200, and/or the position of the movable portion 300.

One measurement set may comprise a plurality of flexion cycles, such as 10, 20, 30, 40, 50, or any number therebetween. In some forms, a measurement set may consist of a fewer or greater number of cycles than those specified above. In one example, the measurement set may consist of 30 cycles, taking approximately 30 seconds to complete. By measuring a plurality of cycles, any changes in condition (e.g. stiffening or loosening) of the joint may be captured such that a resulting determination of rigidity may be made.

In some cases, the patient may progressively relax over an initial phase of the plurality of cycles, potentially leading to inconsistent results across the measurement set. The system may be configured to detect such a change throughout the measurement set and discard or disregard the measurements corresponding to the initial phase. In one example, 30 flexion cycles may be captured in a measurement set, wherein the initial phase (e.g. of first five or ten cycles) may be disregarded in determination of the clinical rating.

The set of measurements may then be received by the processor 5500, and applied as inputs to one or more lookup tables and/or transfer functions used to determine a set of one or more outputs, for example a clinical rating. The processor may also control the movement source 200, as indicated in the example shown in FIG. 5.

The system may be operable in a data collection or a calibration mode. In a calibration mode, the set of measurements may be used by a regression model 5400 to produce the one or more lookup tables and/or transfer functions used to process the input.

An example form of a transfer function is shown below.

A₀F_{f}^{r}+A₁W_{f}^{r}+A₂M_{f}^{r}+A₃M_{f}^{a}+A₄Q_{f}^{a}+A₅Fₑ^{a}+A₆Pₑ^{a}+A₇Wₑ^{a}+P_{f}^{r}(A₈F_{f}^{r}+A₉P_{f}^{a}+A₁₀W_{f}^{r}+A₁₁M_{f}^{r}+A₁₂M_{f}^{a})+A₁₂M_{f}^{r}M_{f}^{a}+Q_{f}^{r}(A₁₄W_{f}^{r}+A₁₅W_{f}^{a}+A₁₆M_{f}^{r})+Q_{f}^{a}(A₁₇P_{f}^{r}+A₁₈M_{f}^{r})+Fₑ^{r}(A₁₉P_{f}^{r}+A₂₀P_{f}^{a}+A₂₁W_{f}^{r}+A₂₃M_{f}^{r}+A₂₄Q_{f}^{r})+Fₑ^{a}(A₂₅M_{f}^{r}+A₂₆Q_{f}^{r}+A₂₆Q_{f}^{a})+Pₑ^{r}(A₂₇P_{f}^{r}+A₂₈M_{f}^{a})+Pₑ^{a}(A₂₉M_{f}^{r}+A₃₀Fₑ^{r})+Wₑ^{r}(A₃₁F_{f}^{r}+A₃₂W_{f}^{r})+Wₑ^{a}(A₃₃P_{f}^{a}+A₃₄W_{f}^{a}+A₃₅Pₑ^{a})+c

In this example, A refers to a coefficient, c is a constant, r is a superscript identifier for rest condition, a is a superscript identifier for contralateral activation condition, e is a subscript identifier for extension cycle and f is a Subscript identifier for flexion cycle. The variables are F for force rate, P for peak force, W for work, M for peak current and Q for charge.

It will be understood that a number of forms of transfer functions may be suitable.

Some systems have attempted to characterise a joint's rigidity by determining only a force required or torque required to perform a movement of that joint. The results may have been presented in terms of displacement, such as to arrive at a force per displacement graph. However, such measurements may not be able to differentiate between externally and internally induced movements of the joint. In other words, a clinician looking to characterise rigidity of an elbow may not be able to distinguish whether the forearm resistance level measured is due to the system moving the forearm, patient-initiated movement/effort of the forearm, or some mix thereof. Similarly, a clinician looking to characterise a wrist rigidity may not be able to tell how much of the wrist rigidity measured may be due to internally-induced (i.e. user-induced) movement, such as the patient (consciously or subconsciously) moving their hand. As a result, such a measurement may produce measures of rigidity that may not be adequately representative of a condition of the joint, in turn potentially leading to an inadequate diagnosis and/or titration for treatment.

In some embodiments, a system according to the present disclosure combines at least a first data set indicative of a force applied by a first body region, and a second data set indicative of a torque applied between the first body region and the second body region to determine an improved measure of rigidity of the joint between the first body region and the second body region. The first data set may be from the force transducer 5010, and the second data set may be from the current transducer 5020.

The system may be configured to discard or reject data measured by the set of one or more sensors based on certain criteria, such as to improve accuracy.

For example, a user may begin to subconsciously assist the clinician performing a characterisation test of second finger metacarpophalangeal rigidity, by voluntarily moving the second finger. Such a change may be detected by a marked reduction in force seen in the first data set indicative of the force, while little or no changes may be observed in the second data set indicative of the torque. Data obtained from such trials may be discarded. Alternatively, if the change is detected in real-time, the trial can be rejected and repeated immediately. Thus, the combination of the two data sets may produce a more reliable characterisation of joints.

In another example, one of the data sets could be used to complement, modify or compensate another of the data sets. For instance, the force data set may be used to produce a modified torque data set, arriving at a higher-accuracy characterisation than otherwise possible with only one torque or force data set.

The processor may produce an output data set using the input data sets, the output data set including one or more data subsets and/or points therein, such as to be used in an aggregate model of descriptors of joint rigidity.

The subsets and/or data points may include one or more of a force rate, a peak force, a work, a peak current and/or a charge. A force rate may be defined as a mean force per unit (e.g. degree) required to flex a joint, resulting for example in a Newtons per degree output. A peak force may be a maximum of the measured force throughout the duration of the movement, measured for example in Newtons. A work (e.g. in units of Newton metres or J) may capture an integral of force with respect to displacement (otherwise defined as ‘total area under the curve’ when plotting the force in terms of displacement). A peak current (as a surrogate for torque), similarly to peak force, may measure a maximum of the measured current throughout the duration of the movement, measured for example in Amperes. A charge is analogous to work, measuring an integral of current over time, and measured for example in Coulombs or Ampere-hours.

It will be readily understood by those skilled in the art that the exact units of the above may be varied or scaled, and surrogate measures may be readily available for one or more of the measures or data above.

The output data set may then be compared to a predetermined scale, or be input into a model, to characterise the joint.

In one example, the output data set may be further processed to arrive at a clinical rating 5900 as shown in the example block diagram of FIG. 5, wherein the clinical rating 5900 is scaled on a 0-4 scale based on the UPDRS, however allowing for improved precision and reproducibility in comparison to the above-outlined limitations.

The system may allow characterisation of a joint using an output set including one or more of: a force rate, peak force, work, peak current and charge, determined based on outputs of the force sensor and the torque sensor. The output set, whether used as described above, or as further inputs to a single clinical rating, represents an improved characterisation in comparison to the prior art. This may be due to improved measurement fidelity, and ability to separate internally-induced movement or resistance from the joint rigidity in the joint's relaxed state.

In some forms, the system may comprise one or more predetermined lookup tables or predetermined transfer functions (such as those produced using a regression model based on validation data) to produce a set of outputs, such as a clinical rating, or any other suitable outputs as described throughout the present disclosure. The system may further comprise memory (e.g. non-transient memory) wherein the one or more lookup tables and/or the transfer functions may be saved for retrieval by the processor. The lookup tables and/or transfer functions may be saved in one or more locations, for example on the hand-held device 5800 or on a remote computing device external to the system.

The processor may receive a set of signals from the set of sensors, which may be used as a set of inputs for the one or more lookup tables and/or transfer functions to produce the set of outputs. It may also be possible for the regression models and/or the transfer functions to be redetermined or updated based on collection of further data.

In other embodiments, one or more signals may be used as stand-alone measures for continuous quantitative measurement of joint rigidity. For example, the force rate, peak force, work estimate or charge may each be used individually to provide a stand-alone measure of joint rigidity.

Embodiments of the present disclosure may be potentially used in a variety of settings. For instance, in a clinic setting, an objective measurement of rigidity can be readily made to assess disease severity without physical manipulation of the patient's limbs by the clinician.

Alternatively, or additionally, the patient may be able to make self-measurements. For example, the patient may take daily measurements at home to track disease progression and efficacy of new interventions. In this example, the results may be reported back to the clinician for remote monitoring and follow-up.

Still further, such a system may be used in instances where multiple measurements are required over a period of time, such as during surgery or clinical trials. For example, during deep brain stimulation surgery where electrode position is verified by microelectrode stimulation of the target region to ameliorate PD symptoms, such a system could be utilised for its reproducible, objective outputs. In clinical trials, production of objective measures in increased resolution may be of use.

Some forms of the systems may further comprise a controller, such as for a clinician to perform the study remotely from the patient or the user. The system may comprise one or more wireless communication capabilities, such as to transmit data or to control the system.

In alternative embodiments, the systems, devices and methods may be adapted for monitoring and/or characterising other joints, such as a wrist, elbow, ankle, knee or otherwise.

Moreover, in alternative embodiments, system or devices of the present disclosure may be configured or adapted to deliver an output signal relating to one or more of tremor, bradykinesia or postural instability.

Output signals indicative of tremor may be extracted from raw data from the sensors, for example, by applying filtering strategies. Identified tremor signals may be characterised by amplitude, velocity and acceleration as well as peak frequency and power spectral density. As examples, four types of tremor may be quantified, while wearing the device, using the following methods:

A) resting tremor: patient rests hands on a supportive surface (table or lap) and relaxes muscles;

B) postural tremor: patient holds arms in a position against gravity (e.g. arms outstretched);

C) kinetic tremor: patient performs repeated voluntary movements while holding arms in a posture against gravity (e.g. repeated elbow flexion and extension); and

D) intention tremor: patient performs repeated voluntary reaching tasks towards a target (e.g. asking the participant to perform the “finger-nose” exercise, that is, alternate between reaching for the participant's nose and the examiner's finger held approx. 1 m away from the subject).

Bradykinesia (slowness of movement) is conventionally characterised in a clinic through observation of repeated movement such as finger tapping or wrist rotation. The amplitude, speed, and consistency of movement are noted by the clinician. The inertial motion unit of embodiments of the system may be used to obtain measurements related to such movement. To assess bradykinesia using embodiments of the system, the participant may be asked to repeatedly rotate their wrist between palm-up and palm-down positions (pronation/supination) while wearing the device. The motion data generated by the system may then be analysed to determine amplitude, velocity and acceleration as well as decay in the rate of movement over time and the number of cycles performed per second (frequency). These metrics may be used to determine bradykinesia severity.

Postural instability may also be assessed using a system according to embodiments of the present invention. While wearing the device on his/her hand, a patient may be asked to cross his/her arms on the chest. The system may then be used to monitor movement of the trunk to ascertain a surrogate measure for centre-of-mass. Postural instability may be assessed under two conditions:

A) Static balance: patient standing upright in quiet comfortable stance with eyes open and fixated on a wall feature (e.g. painting). Trunk movements during this task may be recorded using the system for a period of time, for example, 30 seconds. Subtle movements in centre-of-mass (sway) occur to keep balance. Similar to intoxication-induced loss of balance coordination, the extent and pattern of sway can be used to characterise disease. Postural sway generally occurs in a frequency range between 0.01-2 Hz, thus extraneous motion from tremor occurring in higher frequency bands can be attenuated. Postural sway may be quantified by traditional metrics such as: the area of an ellipse which covers 90% of the sway data (C90 Area), sway range/magnitude, peak frequency, and power spectral density. Furthermore, computational modeling using data generated by embodiments of the system may be used to deduce and quantify deficiencies in feedback mechanisms (e.g. Proportional-Integral-Derivative (PID) controller with an inverted pendulum modeling human stance).

B) Dynamic balance: patient standing upright in a quiet, comfortable stance and an examiner, standing behind the patient, applies a brisk backwards pull on the patient's shoulders. This is a conventional clinical examination, referred to as the pull test, used to observe and assess balance reflexes. These balance reflexes, such as trunk and foot reflexes, may be quantified using embodiments of the system by setting signal thresholds to detect onset of the applied pull and trunk/foot reflex responses. The timing and magnitude of responses may be used to detect and quantify deficiencies in balance.

### EXAMPLE

A validation study of aspects of the present disclosure has been carried out, and is described in detail below.

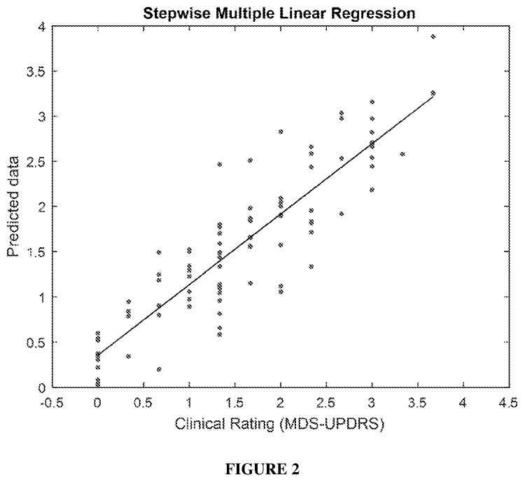

The study was based on data collected from participants in both a relaxed state, and while performing an ‘activation task’ (i.e. typically being a contralateral hand movement which increases rigidity on the resting side). By applying the output data set described above to both the resting and activated trials and performing a step-wise multiple linear regression, it was found to be possible to model the rigidity and match the clinical ratings with satisfactory congruence. The model was verified, for example using a stratified 10-fold cross validation with 50 iterations, and residuals are normally distributed. FIG. 2 shows one validation result set, comparing a set of clinician-produced MDS-UPDRS data (horizontal axis) to those of predicted data (vertical axis).

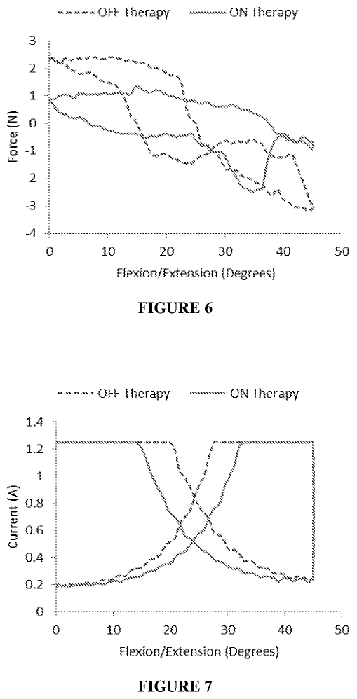

FIGS. 6 and 7 show example outputs of measurements taken during the study. Each of these examples show data from a single patient, one difference between each of the traces being whether a therapy (deep brain stimulation) is being applied to the patient. Deep brain stimulation (DBS) can affect rigidity in the patient's joint. This change is shown in FIGS. 6 and 7. In both figures, dotted lines show results without therapy, and solid lines show results while the therapy is being applied.

### Methods

Eight participants (mean standard deviation age 53.6 5.7 years; 2 female) diagnosed with levodopa-responsive Parkinson's disease receiving bilateral subthalamic nucleus deep brain stimulation (DBS) were recruited as a primary study cohort. Age-matched healthy volunteers (N=8, aged 54.1 7.3 years; 2 female) and young healthy subjects (N=8, 30.9 4.8 years; 2 female) served as controls. Participants had no known musculoskeletal or other neurological disorders (other than Parkinsonian symptoms for that respective cohort).

A system according to an embodiment of the present disclosure was used to characterise joint rigidity for this study. This embodiment includes a palm-worn device as illustrated in FIGS. 3 and 4, which flexes the second finger of the hand about the metacarpophalangeal joint. A miniature on-board electric motor automatically flexes and extends the digit. An increase in rigidity required greater force to flex the digit.

In the system utilised for the study, a force transducer mounted beneath the finger harness measures flexion and extension force. Additionally, an electronic sensor monitors current drawn by the motor as a surrogate for torque. Generally, motor current is linearly proportional to the torque output of the motor. The motor of this embodiment has integrated gearing with sufficient torque to flex the finger by 45-degrees at a continuous rate of one extension/flexion cycle per second. Further, the motor has internal feedback control allowing precise positioning. A microprocessor actuates the motor and gathers time-stamped motor position (displacement), force, and current data at 250 samples/s which is then transmitted to a computer for offline analysis.

Participants were familiarised with the palm-worn device and a practice trial was conducted prior to formal assessment. Each rigidity assessment consisted of 15 continuous extension/flexion cycles applied without removing the palm-worn device. There were no hold periods where movement did not occur. The first five cycles were discarded to avoid confounds from oppositional paratonia (involuntarily resisting movement) and any startle effects. The entire assessment was rejected if abnormally low force (<10 mN) was detected in more than 50% of the remaining cycles. This removed trials where facilitatory paratonia (involuntarily assisting passive movement) may have been present. In each extension/flexion cycle, periods relating to movement onset and change in direction were excluded to avoid confounds of inertial force. Any baseline offset was subtracted. The following features were extracted from the average of the ten cycles separately for extension and flexion:

1) Force Rate—the slope of the force vs. displacement curve expressing force per degree required to move the finger (FIG. 6)

2) Peak Force—the maximum of the force vs. displacement curve

3) Work Estimate—the total area under the force vs. displacement curve

4) Charge—the total area under the current vs. time curve (FIG. 7).

To deduce a measure of rigidity that is continuous and quantifiable, the flexion Force Rate and Work Estimate were selected to be used in subsequent analysis as stand-alone measures of rigidity. These metrics have meaningful units and are easy to interpret with a higher value indicating greater rigidity.

Participants with Parkinson's disease were assessed using conventional clinical assessment. As per the instructions in the MDS-UPDRS Item 3.3, assessment of rigidity for the upper extremities was performed by slow movement of the limp wrist and elbow joints. Participants were seated comfortably in a relaxed state and were instructed to make their body as relaxed as possible. For the activation manoeuvre, participants were instructed to draw a large imaginary circle in the air using their contralateral arm. Neck and lower extremities were not examined. Two physiotherapists (“raters”), experienced in movement disorders and the MDS-UPDRS, rated rigidity on a scale of 0 (normal) to 4 (severe) as per descriptors in the MDS-UPDRS. Both raters were blinded to the protocol and directed not to discuss their ratings with each other until the conclusion of the study. Raters were only present in the assessment area to perform ratings. The mean clinical rating from the two physiotherapists was used in subsequent analysis.

Participants with Parkinson's disease arrived at the clinic on-DBS and off-dopaminergic medication following overnight withdrawal. Rigidity of both arms was measured first using the system including the palm-worn device, then by the two raters in quick succession at baseline (on-DBS), and at 10-min intervals following therapy withdrawal (off-DBS) over a one-hour period. Rigidity was then assessed by the system alone at 5-min intervals following DBS resumption over a 30-min period. At the last trial within this period, the two raters also assessed rigidity. This protocol sought to monitor the wash-out and -in effects of DBS.

All healthy volunteers were assessed with the palm-worn device and no clinical examination was conducted. The dominant hand of each individual was assessed while they were at rest or performing a contralateral activation task (as described previously). Ten trials (5 rest, 5 activation) were performed in random order. Participants were seated and asked to make their body as relaxed as possible.

### Statistical Analysis

A two-way repeated-measures analysis of variance (RM-ANOVA) was conducted to determine if DB S therapy and the contralateral activation task influenced rigidity. Post-hoc paired t-tests were applied to further define any differences. A two-way ANOVA was conducted to deduce if there were differences between healthy volunteers (coded for both age-matched and young) and those with Parkinson's disease as well as any influence arising from the activation task. Post-hoc independent t-tests were performed to evaluate any specific differences within cohorts. Cohen's d was calculated for each post-hoc comparison to indicate effect size. Values of d can be interpreted as the effect size being: very small (0.01), to medium (0.5), large (0.8), and huge (2.0).

Cohen's Kappa (κ) was used to determine inter-rater reliability. Test-retest reliability was calculated using two-way random single measures intraclass correlations (ICCs) for both the system including the palm-worn device and clinical scores using data from the first assessment at baseline and the last assessment following DBS resumption.

### Results

Overall, eight clinical ratings were recorded across both hands in eight Parkinson's participants resulting in a dataset of 128 observations. Participant 4's left hand data (8 observations) were excluded due to mechanical failure of the device. Participant 5's left hand data (8 observations) were excluded due to limited range of movement in finger joints. A further 22 observations were removed due to facilitatory paratonia (force<10 mN detected), bringing the total number of observations to 90. Inter-rater reliability between the two examiners was fair. Test-retest reliability for the clinical scores, Force Rate, and Work Estimate were good.

Congruence with MDS-UPDRS and Predictive Features

A model resulting from the stepwise linear regression was developed. Comparison between predicted estimates and clinical ratings showed the model performed moderately well. Stratified 10-fold cross validation resulted in only a marginal decrease in performance suggesting the model did not overfit the data. Moreover, the model residuals were normally distributed indicating no underlying pattern or occurrence of outliers. The terms which had the greatest influence in the model were: Force Rate during finger flexion with hands at rest Peak Force during extension with activation task, Work during flexion with activation task, and the interaction between Force Rate and Peak.

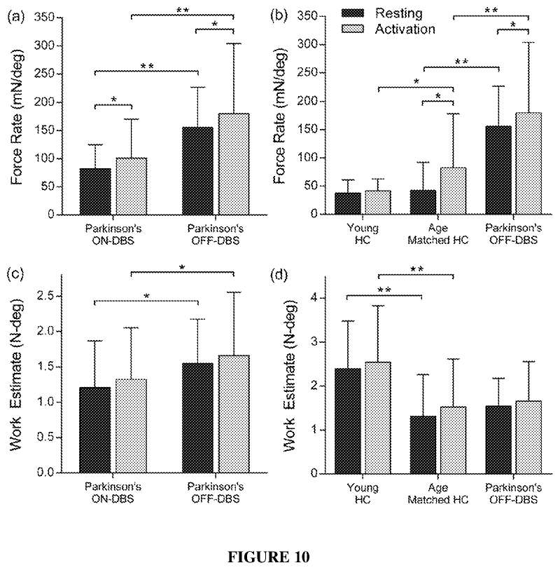

In subsequent analysis two metrics (Force Rate and Work Estimate during finger flexion) were used to characterise rigidity. Though these metrics alone had relatively low congruence with clinical ratings, they offer quantifiable measures of rigidity on a continuous scale. Importantly, both metrics emerged as important terms in the stepwise linear regression analysis. Force Rate can be used to differentiate therapeutic states and characterise disease, as shown in FIG. 10. A two-way RM-ANOVA indicated a statistically significant main effect for DBS setting (on vs. off) and test condition (rest vs. activation). Post-hoc tests revealed that the absence of DBS therapy resulted in greater Force Rate and a contralateral activation exercise led to an increase in Force Rate both on and off DBS (FIG. 10a). A two-way RM-ANOVA indicated that Work Estimate can be used to differentiate therapeutic states, but not rest vs. activation. Post-hoc tests revealed that DBS therapy significantly reduced Work Estimate.

### Ability to Differentiate Healthy and Parkinsonian Cohorts

A two-way ANOVA for Force Rate revealed a statistically significant main effect for cohort (Parkinson's disease off-DBS vs. age-matched controls vs. young controls) and test condition (rest vs. activation). Post-hoc tests revealed a significant increase in Force Rate due to Parkinson's disease when compared to age-matched controls during both rest and activation tasks (FIG. 10b). Age-matched controls had a higher Force Rate than their younger counterparts during the activation task, but no difference in resting Force Rates. Also, contralateral activation compared to resting led to an increase in Force Rate in the age-matched group, but not in young controls. DBS therapy in the Parkinson's cohort decreased Force Rate towards levels found in age-matched controls suggestive of therapeutic effect, yet a significant difference remained in the resting condition.

A two-way ANOVA indicated that Work Estimate can be used to differentiate study cohorts, but not rest vs. activation conditions. Post-hoc tests revealed no significant difference in Work Estimates between age matched controls and those with Parkinson's disease. However, age-matched controls had a lower Work Estimate compared to younger volunteers.

### Transient Effects of DBS Wash-In/Out

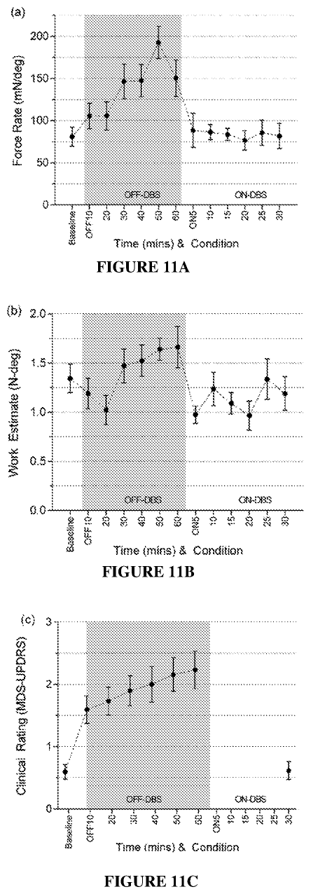

The transient wash-in/out effects of DBS were documented using Force Rate, Work Estimate and clinical ratings, as shown in FIGS. 11A to 11C, respectively. DBS cessation led to a steady increase in rigidity over a one-hour period, and DBS resumption resulted in an almost immediate improvement in rigidity back towards baseline levels. Work Estimate displayed greater variability than Force Rate.

### Discussion

In this feasibility study, it was found that the system including the palm-worn device had moderate agreement with clinical rigidity ratings and was able to distinguish differences between therapeutic conditions, contralateral activation exercises, as well as participants with and without Parkinson's disease. Though a multitude of metrics derived from the device were used in the regression model to predict clinical ratings, a single metric (Force Rate) was sufficient to provide further characterisation. It was found that Force Rate, the amount of force required per degree of finger flexion, increased over the period of one hour when DBS was ceased. Resuming DBS therapy decreased Force Rate back to baseline levels within five minutes. Additionally, Force Rate was markedly increased in participants with Parkinson's disease compared to age-matched healthy controls. Although Work Estimate was able to differentiate therapeutic states, it was insensitive to contralateral activation and could not distinguish between age-matched controls and those with Parkinson's disease.

The moderate congruence achieved in the present example is comparable to previous attempts at instrumented quantification of rigidity. The moderate fit and large RMSE suggest that either the system did not capture all aspects of rigidity, or that the clinical assessments had inherent inaccuracies and that the system was more sensitive to fluctuations in resistance to passive motion. Moreover, the moderate relationship may be expected given the clinical and device measures were of two disparate origins (the palm-worn device using the metacarpophalangeal joint and clinicians using the wrist and elbow as per MDS-UPDRS guidelines). Since increased neuronal activity mediates muscle tone in Parkinson's disease, this may be generalisable to all proximal muscles. However, the effects of rigidity may influence each muscle group variably. Sensitivity to detect therapeutic change is of fundamental importance to clinical applications and this has been demonstrated using the palm-worn device.

Elastic, viscous, inertial, and frictional stiffness of muscles are independent components that contribute to the overall rigidity felt as resistance to passive movement during clinical examination. Force Rate primarily quantifies elastic stiffness and is usually presented in units of torque per degree (Nm/deg). The presently disclosed method may not be specific to elastic stiffness as it may detect involuntary muscle reflexes to passive movement; this may also explain the moderate agreement with clinical ratings. Conversion to torque typically entails measurement of the limb segment length under evaluation to determine the distance from a pivot (joint) where force is applied. This measurement is prone to error and must be repeated for each individual. In the present example, the finger harness was designed to standardise the distance at which force was applied to pivot the metacarpophalangeal joint, such that the force results can easily be converted to torque via simple scaling.

In this study, it was found that Force Rate increased as a result of therapy, contralateral activation, and disease (Parkinson's vs. controls). This is in accordance with previous work reporting that torque-angle slopes typically become steeper with worsening rigidity.

Work Estimate or the “Rigidity Work Score” is often presented with units (N-deg) or normalised by the range of motion (N-deg/deg). Work Estimate has been previously shown to differentiate controls and those with Parkinson's disease as well as contralateral activation and rest. The results of this study do not support these findings possibly due to a number of methodological differences. Notably, comparable studies used either the wrist or elbow joints to determine Work Estimate. Moreover, they adopted a greater range of motion and slower movement speed than the present study, with both factors known to influence rigidity assessment.

Previous studies investigating rigidity in the metacarpophalangeal joint have utilised benchtop mounted instruments requiring arms to be fixed to large contraptions. Motorised techniques required large powerful motors to generate adequate torque to displace limbs. Importantly, limb segments require bracing against support surfaces to constrain movement to a single joint and axis. The palm-worn instrument utilised in this example incorporates a miniature motor with geared output to drive the finger, the base of the instrument acts as the support surface and is tethered to the palm of the hand. This leads to minimal restriction of joint movement. Consequently, raters in the present study were able to carry out the standard MDS-UPDRS assessment on the wrist and elbow without removing the device from the palm. Furthermore, participants were not tethered to a large instrument and were free to move their arms during periods of rest.

The findings support the notion that an instrumented rigidity measure offers advantages over conventional clinical ratings. It may be used to guide DBS surgery and assess intraoperative effects of stimulation, determine optimum therapeutic windows, and provide insight into mechanism of therapeutic action. Importantly, instrumented methods allow continuous monitoring, delivering insight into temporal characteristics of symptom changes.

This example demonstrates that the disclosed system can quantify finger rigidity in Parkinson's disease. The system can track rigidity over time in moderate agreement with clinical observations. Importantly, the capability to detect changes arising from therapeutic intervention may prove useful in clinical trials or as a home-based monitoring tool to track symptom fluctuations.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A system for characterising a rigidity of a joint between a first body region and a second body region, the system comprising:
a reference portion configured to be coupled to the first body region;
a movable portion configured to be coupled to the second body region and to allow the second body region to move relative to the first body region in a first direction when coupled thereto;
a movement source configured to move the movable portion relative to the reference portion in the first direction;
a set of sensors comprising:
a first sensor configured to determine a force between the movable portion and the second body region;
a second sensor configured to determine a power consumed by the movement source; and
a processor,
wherein the processor is configured receive a set of signals from the set of sensors indicative of determinations from its sensors and to deliver an output signal indicative of the rigidity of the joint based at least on the set of signals.

2. The system of claim 1, wherein the joint is a metacarpophalangeal joint.

3. The system of claim 1, the set of sensors further comprising a third sensor configured to determine a position of the movable portion relative to the reference portion.

4. The system of claim 1, the set of sensors further comprising a fourth sensor configured to determine inertial motion of the system.

5. The system of claim 1, wherein the output signal comprises a clinical rating.

6. The system of claim 5, wherein the processor determines the clinical rating based on a lookup table.

7. The system of claim 1, wherein the output signal comprises one or more of: a force rate, peak force, work, peak current and charge.

8. The system of claim 1, wherein the processor is configured to discard a portion of the set of signals based on predetermined criteria.

9. The system of claim 8, wherein the predetermined criteria includes a threshold rate of change of output of the first sensor.

10. A method for determining a rigidity of a joint between a first body region and a second body region for characterisation of the joint, the method comprising:
providing a reference portion coupled to the first body region;
providing a movable portion coupled to the second body region;
providing a movement source configured to move the movable portion relative to the reference portion in a movement direction of the joint;
performing a movement of the first body region relative to the second region in the movement direction, wherein a set of sensors deliver a set of sensor outputs indicative of a force between the movable portion and the second region and a power consumed by the movement source; and
determining, using a processor, a rigidity of the joint at least partly based on the sensor outputs.

11. The method as claimed in claim 10, wherein the joint is a finger joint.

12. The method as claimed in claim 10, wherein the rigidity of the joint is determined using a lookup table.

13. The method as claimed in claim 10, wherein the movement comprises a plurality of flexion cycles of the joint.

14. (canceled)

15. The method as claimed in claim 10, wherein the joint rigidity is determined as a clinical rating.

16. The method as claimed in claim 10, wherein the set of sensor outputs are further indicative of tremors occurring on the joint.

17. The method as claimed in claim 10, wherein the set of sensor outputs are further indicative of a position of the movable portion.

18. (canceled)

19. The method as claimed in claim 10, wherein the movement is at a constant speed.

20. A device for characterising a rigidity of a joint between a first body region and a second body region, the device comprising:
a reference portion configured to be coupled to the first body region;
a movable portion configured to be coupled to the second body region and to allow the second body region to move relative to the first body region in a first direction when coupled thereto;
a movement source configured to move the movable portion relative to the reference portion in the first direction;
a first sensor configured to determine a force between the movable portion and the second body region; and
a second sensor configured to determine a power consumed by the movement source.

21. The device of claim 20, wherein the first sensor is located on a surface of the movable portion.

22. The device of claim 20, wherein the movable portion is configured to be coupled to a finger and the reference portion is configured to be coupled to a palm.
